(19) United States
(12) Patent Application Publication
(10) Pub. No.: US 2009/0131504 A1
(43) Pub. Date: May 21, 2009

(54) USE OF MELATONIN-CONTAINING ORAL SUPPLEMENT WITH ANIMALS

(76) Inventor: John G. GORDON, New Albany, OH (US)

Correspondence Address: STANDLEY LAW GROUP LLP, 6300 Riverside Drive, Dublin, OH, 43017, US

(21) Appl. No.: 12/355,844

(22) Filed: Jan. 19, 2009

Related U.S. Application Data
(63) Continuation of application No. 10/902,194, filed on Jul. 29, 2004, now abandoned.
(60) Provisional application No. 60/494,819, filed on Aug. 13, 2003.

(51) Int. Cl. A61K 31/4045

(52) U.S. Cl. 514/415

(57) ABSTRACT

The diet of a healthy non-human animal is supplemented with a melatonin-containing supplement by providing a dietary supplement containing melatonin in an amount from about 0.1 mg to about 12 mg/kg of body weight of the animal and administering the dietary supplement to the animal at a regular interval. The selected interval may be daily, weekly, monthly, or seasonally. The melatonin-containing supplement is in the form of a pill, a tablet, a capsule, a powder, a liquid, or is incorporated into food or food pellets. The method is practiced on cats and dogs.

### CROSS-REFERENCE TO RELATED APPLICATIONS

This application is a continuation of U.S. application Ser. No. 10/902,194 filed Jul. 29, 2004, now abandoned, which claims the benefit of U.S. Provisional Application Ser. No. 60/494,819, filed on Aug. 13, 2003, each of which is incorporated herein by reference.

### BACKGROUND AND SUMMARY OF THE INVENTION

The present invention relates to an oral supplement containing a low dosage or small amounts of melatonin. The melatonin-containing oral supplement may be provided to animals for the regulation or control of shedding and/or for the enhancement of the hair coat of the animal.

Melatonin is a hormone synthesized in the pineal gland. Melatonin is secreted in response to low light intensity and decreasing duration of daylight. Melatonin has known uses in humans for sleep disorders, jet lag syndrome, male pattern alopecia, mental depression, anti-aging, use as an antioxidant, contraception, and stress relief. Some of melatonin's veterinary uses are for the neuroendocrine control of photoperiod-dependent molting and/or pelage color in mammals, fur growth in mink and foxes (and in other species raised for fur production), modulation of reproduction for those species which are seasonally reproductive, and medically for the re-growth of hair in canines with recurrent flank alopecia or pattern baldness. Most recently melatonin products have emerged that tout the use for calming stressed, nervous or anxious pets and as a sleep aid for pets.

The present invention is an oral supplement containing a small amount of melatonin. The oral supplement may be used with domestic animals, horses, zoo animals, livestock or farm animals and may be especially preferred for use with dogs and cats. The melatonin-containing oral supplement may regulate or control the amount of shedding by the animal and the supplement may enhance the hair coat of the animal by providing a fuller, thicker and healthier appearing coat. The melatonin-containing oral supplement may be administered to the animal at regular intervals.

The common hair cycle is divided into three stages: anagen, catagen and telogen. During the active phase (anagen), the epidermal stem cells of the dermal papilla divide rapidly. Daughter cells move upward and differentiate to form the concentric layers of the hair itself. The transitional stage, catagen, is marked by the cessation of mitosis of the stem cells in the follicle. The resting stage is known as telogen, where the hair is retained within the dermis for several weeks before an emerging new hair developing below it dislodges the telogen-phase shaft from its follicle.

The present invention is an oral supplement for the regulation or control of shedding in animals. The supplement may be in pill form, in tablet form, in liquid form, in powder form, in capsule form, or may be mixed into food or food pellets. The supplement may given or administered at regular intervals.

The present invention is a method of treating animals with a melatonin-containing oral supplement for the regulation or control of shedding and for the enhancement of the animal's hair coat. In one preferred embodiment, the present invention may be specifically directed for use with cats and dogs.

In addition to the novel features and advantages mentioned above, other features and advantages of the present invention will be readily apparent from the following descriptions of the drawings and exemplary embodiments.

### DETAILED DESCRIPTION OF EXEMPLARY EMBODIMENT(S)

The present invention is a method of treating animals with a small amount of a melatonin-containing oral supplement for the regulation or control of shedding and for the enhancement of the animal's hair coat. The term “small amount” in reference to the amount of melatonin may vary depending on the weight or the body surface area of the animal being treated. Livestock, farm animals, horses or zoo animals may require substantially larger regular amounts of melatonin than domestic animals. Melatonin is preferably contained in veterinary oral supplements in the range of from about 0.1 mg to about 12 mg/kg of body weight. Most preferably melatonin is in the range of from about 0.5 mg to about 1.5 mg/kg of body weight. The amount of melatonin given may be dependent on how often it is given. If the supplement is administered multiple times per day the amount may be lower than if administered on a daily, weekly, monthly or seasonal basis. Melatonin may be purchased from Sigma Chemical Company (St. Louis, Mo.).

The oral supplement of the present invention may be in pill form, in tablet form, in capsule form, in powder form or may be a liquid or may be incorporated into food or food pellets. The oral supplement is used with animals and is especially preferred for use with domestic animals, horses, livestock, farm animals, and zoo animals and most preferably used with dogs and cats. The melatonin-containing oral supplement may be given at regular intervals. For example, the supplement may be given on a daily, weekly, monthly, or seasonal basis or at another regular interval of time.

The oral supplement of the present invention may be given to animals to regulate or control shedding. Regulation or control of shedding may mean overall shedding is reduced, overall shedding is normalized, shedding is forced to occur at sustained rates throughout the year or that dramatic shedding is lessened.

The oral supplement of the present invention may be given to animals to enhance the hair coat of the animal by providing a fuller, thicker, and healthier appearing coat of hair.

The exemplary embodiments herein disclosed are not intended to be exhaustive or to unnecessarily limit the scope of the invention. The exemplary embodiments were chosen and described in order to explain the principles of the present invention so that others skilled in the art may practice the invention. The dosage and frequency of administration of the melatonin containing supplement may be varied in numerous ways and all these variations fall within the scope of the present invention. Having shown and described exemplary embodiments of the present invention, those skilled in the art will realize that many variations and modifications may be made to affect the described invention. Many of those variations and modifications will provide the same result and fall within the spirit of the claimed invention. It is the intention, therefore, to limit the invention only as indicated by the scope of the claims.

What is claimed is:

1. A method of supplementing the diet of a healthy, non-human animal with a melatonin-containing supplement comprising:
providing a dietary supplement containing melatonin in an amount from about 0.1 mg to about 12 mg/kg of body weight of the animal; and
administering the dietary supplement to the animal at a regular interval.

2. The method of claim 1, wherein the animal is selected from the group consisting of domestic animals, farm animals and livestock.

3. The method of claim 2, wherein the animal is selected from the group consisting of cats and dogs.

4. The method of claim 1, wherein the regular interval is selected from the group consisting of daily, weekly, monthly and seasonally.

5. The method of claim 1, wherein the melatonin-containing supplement is in the form of a pill, a tablet, a capsule, a powder, a liquid, or is incorporated into food or food pellets.

6. The method of claim 1, wherein the melatonin-containing supplement is incorporated into the animal's food.

7. The method of claim 1, wherein the amount of melatonin in the melatonin-containing supplement is from about 0.5 mg to about 1.5 mg/kg of body weight.

8. A method of supplementing the diet of a healthy non-human animal with a melatonin-containing supplement comprising:
providing a dietary supplement containing melatonin in an amount from about 0.1 mg to about 12 mg/kg of body weight of the animal; and
administering the dietary supplement to the animal at a regular interval;
wherein the regular interval is chosen from the group consisting of daily, weekly, monthly, and seasonally;
wherein the animal is chosen from the group comprising cats and dogs; and
wherein the melatonin-containing supplement is in the form of a pill, a tablet, a capsule, a powder, a liquid, or is incorporated into food or food pellets.
